(19) United States
(12) Patent Application Publication
(10) Pub. No.: US 2008/0287491 A1
(43) Pub. Date: Nov. 20, 2008

(54) Tetrahydroquinoline Derivative for Treating Nicotine Craving

(76) Inventors: Cristiano Chiamulera, Verona (IT); Angelo Reggiani, Verona (IT); David Trist, Verona (IT); Vincenzo Teneggi, Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 10/596,132

(22) PCT Filed: Nov. 30, 2004
(86) PCT No.: PCT/EP04/13666

§ 371 Date: Jun. 1, 2006

(30) Foreign Application Priority Data
Dec. 2, 2003 (GB) 0327912.2

(51) Int. Cl. A61K 31/4709; A61P 25/34

(52) U.S. Cl. 514/314

(57) ABSTRACT

The present invention relates to a new medical use for a compound of formula (I):

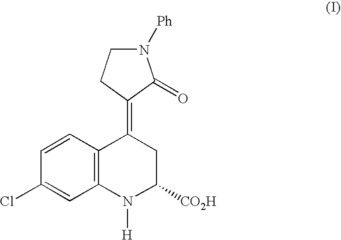

for the treatment of nicotine craving.

This invention relates to the use of a tetrahydroquinoline derivative and pharmaceutically acceptable salts thereof in the treatment or prevention of nicotine craving. Particularly, the present invention relates to the use of (2R,4E)₇-chloro-4-(2-oxo-1-phenyl-3-pyrrolidinylidene)-1,2,3,4-tetrahydro-2-quinoline and pharmaceutically acceptable salts thereof.

International patent application no. WO 99/64411 describes novel tetrahydroquinoline derivatives. A particular preferred compound described therein is 7-chloro-4-(2-oxo-1-phenyl-3-pyrrolidinylidene)-1,2,3,4-tetrahydro-2-quinoline. Particularly, it describes an enantiomer of the compound of formula (I), which is referred therein as enantiomer A, and a sodium salt thereof which may be represented by the formula (I),

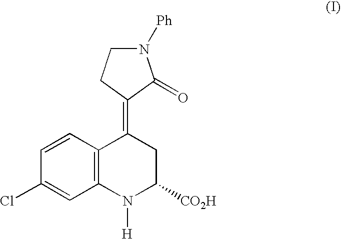

wherein the broken shaped bond indicates that the bond is under the plane of the paper. The aforementioned specification also discloses pharmaceutically acceptable salts and solvates, e.g hydrates of the compound of formula (I).

Suitable pharmaceutically acceptable salts of the compound of general formula (I) include base addition salts of compounds of formula (I) such as sodium, potassium, calcium, magnesium and ammonium salts, formed with amino acids (e.g. lysine and arginine) and organic bases (e.g. procaine, phenylbenzylamine, ethanolamine diethanolamine and N-methyl glucosamine).

International patent application WO 01/42238 describes the meglumine salt of the compound of formula (I) and particularly discloses the meglumine salt of the compound of formula (I) in a crystalline form.

The compound of formula (I) and salts thereof are described in the aforementioned specification as particularly potent antagonists of the NMDA receptor complex. More particularly, the compound of formula (I) and salts thereof are potent antagonists at the strychnine insensitive glycine binding site associated with the NMDA receptor complex.

By virtue of their efficacy as antagonists of the NMDA receptor complex, the compound of formula (I) and its pharmaceutically acceptable salts and solvates are useful for the treatment of drug dependency, including withdrawal symptoms from alcohol, cocaine, opiates, nicotine (e.g. smoking cessation), benzodiazepines and inhibition of tolerance induced by opioids (i.e morphine).

We have now surprisingly found that the compound of formula (I) and pharmaceutically acceptable salts and solvates thereof reduce craving for nicotine and therefore are also useful in the treatment of nicotine craving.

Nicotine craving can be defined as a motivation to self-administer the psychoactive nicotine substance that was previously consumed. Four main factors can characterise nicotine craving: (1) Expectancy or anticipation of positive outcomes from nicotine consumption (2) Expectancy or anticipation of relief from nicotine withdrawal symptoms or negative mood (3) Compulsivity or inability to control nicotine substance use (4) Planning for nicotine consumption.

Two main factors are considered to be involved in the development and maintenance of nicotine craving: (1) Environmental stimuli (cues) associated with nicotine effects (2) Dysphoric/Stressful states. Both these can become, progressively, more powerful in inducing nicotine craving.

Craving may account the difficulty that individuals have in giving up nicotine-containing products and/or maintaining nicotine abstinence.

A particularly preferred pharmaceutically acceptable salt of the compound of formula (I) for use according to the present invention is the meglumine salt.

Accordingly, the invention provides a method of treatment of nicotine craving which comprises administering to a human or animal subject an effective amount of the compound of formula (I) or a pharmaceutically acceptable salt or solvate thereof.

It will be appreciated that the compound of formula (I) may be used prophylactically and references in this specification to treatment include prophylactic treatment as well as the alleviation of acute symptoms.

Accordingly, the invention also provides a pharmaceutical composition which comprises the compound of formula (I) and pharmaceutically acceptable salts and solvates thereof for the treatment or prevention of nicotine craving.

In a yet further aspect, the invention provides the use of the compound of formula (I) or a pharmaceutically acceptable salt or solvate thereof, for the manufacture of a medicament for the treatment of nicotine craving.

In a yet further aspect, the invention provides the use of the compound of formula (I) or a pharmaceutically acceptable salt or solvate thereof, for the treatment of nicotine craving.

Pharmaceutical compositions for use in accordance with the present invention may be formulated in conventional manner using one or more pharmaceutically acceptable carriers or excipients.

Thus, the compound of formula (I) and its pharmaceutically acceptable salts and solvates may be formulated for oral, buccal, parenteral, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or formulated in conventional manner.

The compound of the invention and its pharmaceutically acceptable salts and solvates may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compound of the invention and its pharmaceutically acceptable salts and solvates may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compound of the invention and its pharmaceutically acceptable salts and solvates may also be formulated in rectal compositions such as suppositories or retention enemas, e.g. containing conventional suppository bases such as cocoa butter or other glycerides.

The compound of the invention and its pharmaceutically acceptable salts and solvates may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compound of the invention and its pharmaceutically acceptable salts and solvates may be formulated as solutions for administration via a suitable metered or unitary dose device or alternatively as a powder mix with a suitable carrier for administration using a suitable delivery device.

A proposed dose of the compound of the invention is 1 to about 1000 mg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

Thus, for parenteral administration a daily dose will typically be in the range of 1 to about 200 mg, preferably 40 to 150 mg per day. For oral administration a daily dose will typically be within the range 1 to 300 mg e.g 1 to 100 mg.

The compound of formula (I) and pharmaceutically acceptable salts thereof may be prepared by the process described in international patent applications no. WO 99/64411 and no. WO 01/42238 which are incorporated herein by reference.

Alternatively, the compound of formula (I) or pharmaceutically acceptable salts thereof may be prepared by stereoselective hydrolysis of a compound of formula (II), wherein R is C1-4 alkyl such as ethyl, using a suitable lipase enzyme according to the scheme1.

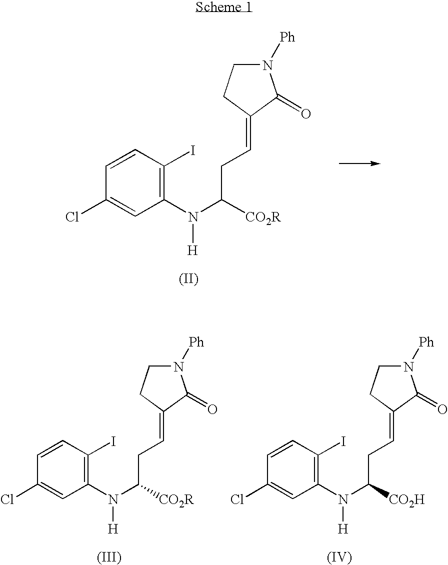

Suitable lipase enzymes for use in this reaction are Mucor miehei lipase or Candida rugosa lipase.

The reaction is conveniently carried out in an organic-aqueous solvent mixture.

Preferably the reaction may be carried out in t-butyl alcohol and water mixture.

The lipase enzyme employed in the present process may be in solution or in an immobilized form. It can be immobilized on various solid supports. The solid support can be inert absorbents to which the enzyme is not covalently bonded. Inert absorbent materials include, but are not limited to, synthetic polymers (e.g. polystyrene, poly(vinylalcohol) polyethylene and polyamides), minerals (e.g. diatomaceous earth and Fuller's earth) or naturally occurring polymers (e.g. cellulose). Specific examples of such materials include Celite 545 diatomaceous earth, Ambelite XAD-8 polymeric resin beads and polyethylene glycol 8000.

The enzyme according to the invention may also be immobilized on the support to which the enzyme is covalently bonded (e.g. oxirane-acrylic beads and glutaraldehyde activated supports). Other possibly immobilizing systems are well known and are readily available to those skilled in the art of the enzyme immobilation.

Particularly useful commercial immobilized enzymes from Mucor miehei lipase are for example Lipozyme or Lipozyme RM (Novo Nordisk) or Chirazyme L9.

Particularly useful commercial immobilized enzymes from Candida rugosa lipase are for example Chirazyme L3, ChiroCLEC CR.

Compound (III) may be converted into compound (V),

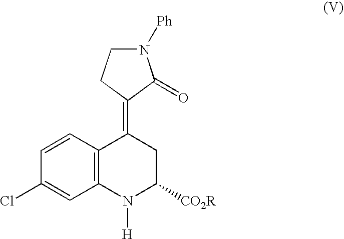

by reaction with a catalytic amount of a Pd(II) salt such as palladium acetate or palladium dichloride in the presence of a suitable organic base such as trialkyl amine e.g. triethylamine and of a triarylphosphine such as triphenylphosphine followed by trimercaptotriazine.

The reaction is carried out in an aprotic solvent such as toluene and preferably with heating. Compound of formula (I) may be obtained from a compound of formula (V) using conventional methods for converting C1-4 alkyl ester into the carboxylic acid followed by reaction with a pharmaceutical acceptable base to form the corresponding pharmaceutically acceptable salt.

The unwanted enantiomer of formula (IV) may be converted into the corresponding C1-4 alkyl ester by conventional means known to transform an acid into an ester and then converted into the racemic compound (II) for re-introduction into the resolution cycle. The racemisation reaction is carried out under standard basic condition such as in the presence of a strong organic base e.g Sodium ethylate in an alcohol solution.

Compound of formula (II) are known compounds as described in WO 99/64411.

In the Intermediates and Example unless otherwise stated:

Melting point was determined using DSC (Differential Scanning Calorimetry) technique.

Proton Magnetic Resonance (NMR) spectra were recorded on Varian instruments at 300, 400 or 500 MHz, on Bruker instrument at 300 MHz, chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at temperature ranging from 25 to 90° C.

The following abbreviations are used in the text TFA=Trifluoroacetic acid; IPA=isopropyl alcohol.

### Intermediate 1

### (+)-Ethyl (4E)-2-[(5-chloro-2-iodophenyl)amino]-4-(2-oxo-1-phenyl-3-pyrrolidinylidene) butanoate

5-Chloro-2-iodoaniline hydrochloride (112 kg) was suspended in toluene (1064 L) and treated with a solution of potassium carbonate (560 kg,) in water (560 L) for 30 minutes at 20-25° C. The phases were separated and the organic phase was washed with water (560 L). Anhydrous magnesium sulphate (84 kg) was added and the solution dried by Dean & Stark method. A solution of ethyl glyoxalate in toluene (118.7 kg,) was then added to the slurry over 30 minutes, maintaining the reflux. Residual solution was washed in with toluene (12 L) and the mixture maintained at reflux for 5 hours and water (8.8 L) was collected in the Dean & Stark trap. After cooling the mixture to −15° C., vinyloxytrimethylsilane (67.2 kg) was added over 10 minutes. Residual reagent was washed in with toluene (10 L) and the temperature at this point was −19° C. Trimethylsilyl triflate (4.59 kg) was added over 4 minutes with the temperature maintained at −15 to −19° C., residual reagent being washed in with toluene (9 L). After stirring for 30 minutes at this temperature the reaction was quenched by the addition of water (560 L). The phases were separated (some magnesium sulphate crystals present) and the organic phase was washed with water (560 L). The organic phase was filtered to remove residual inorganic solids and then concentrated under vacuum, maintaining the temperature below 60° C., down to 780 L.

To a solution of (±)-Ethyl 2-(5-chloro-2-iodoanilino)-4-xobutanoate (194.9 kg) in acetonitrile, (493 L). 1,8-diazabicyclo[5.4.0]undec-7-ene (62.7 kg) was added followed by a line wash of acetonitrile (15 L) and the mixture was stirred at 18-22° C. for 1 hour and 40 minutes to form the phosphorane. The organic phase containing (i)-Ethyl 2-(5-chloro-2-iodoanilino)-4-oxobutanoate, prepared above, was added to the phosphorane solution over 50 minutes followed by a line wash of toluene (19 L). The mixture was stirred for 45 minutes. Water (560 L) was added. After stirring for 15 minutes the phases were separated and the organic phase concentrated to 370 L. The concentrate was diluted with isopropanol (896 L) and the temperature adjusted to 28° C. Seed crystals of the title compound (125 g) were added and the solution stirred at 25-30° C. Crystallisation commenced after 1 hour and 45 minutes. The slurry was cooled to 22° C., stirred at this temperature for 2 hours then cooled and stirred at 0° C. for 1 hour.

The title compound (117.4 kg) was isolated in a filter drier, washed with isopropanol (448 L) and dried under vacuum at 45-50° C. for 23 hours.

¹H-NMR (CDCl₃) δ (ppm) 7.72 (d, J=8.1 Hz, 2H), 7.56 (d, J=9.0 Hz, 1H), 7.38 (t, J=7.9 Hz, 2H), 7.16 (t, J=7.3 Hz, 1H), 6.58 (m, 1H), 6.49 (m, 2H), 4.87 (d, J=7.8 Hz, 1H), 4.26 (m, 3H), 3.86 (t, J=6.8 Hz, 2H), 2.78 (m, 4H), 1.30 (t, J=7.1 Hz, 3H)

### Intermediate 2

### Ethyl (2R,4E)-2-[(5-chloro-2-iodophenyl)amino]-4-(2-oxo-1-phenyl-3-pyrrolidinylidene)butanoate

Intermediate 1 (135 kg) and lipozyme RM IM supported enzyme (27 kg) were stirred in 88% w/w tert-butanol in water (1620 L) at 40-41° C. for 15 hours.

The resin was removed by filtration and washed with 88% w/w tert-butanol in water (135 L). The filtrates were combined and 2.6% w/w sodium bicarbonate solution (415 kg), taken from a stock solution of sodium bicarbonate (43.2 kg) and water (1620 L), was added whilst maintaining the temperature at 38-40° C. The mixture was stirred at this temperature for 1 hour and the product crystallised. The slurry was cooled to 4° C. over 2.5 hours and stirred at 2-3° C. for 1 hour. The title compound was isolated in a centrifuge and was washed with a mixture 88% w/w tert-butanol (135 L) and water (135 L). The title compound was dried in vacuo at 40-45° C. for approximately 18.5 hours to obtain 57.1 kg

¹H-NMR (CDCl₃) δ (ppm) 7.72 (d, J=8.6 Hz, 2H), 7.57 (d, J=8.8 Hz, 1H), 7.38 (t, J=7.8 Hz, 2H), 7.16 (t, J=7.1 Hz, 1H), 6.58 (m, 1H), 6.49 (m, 2H), 4.87 (d, J=8.1 Hz, 1H), 4.26 (m, 3H), 3.87 (t, J=6.8 Hz, 2H), 2.78 (m, 4H), 1.30 (t, J=7.1 Hz, 3H)

### Chiral HPLC Column Chiralpak AD, 250×4.6 mm.

Mobile Phase 0.05% TFA, 60% IPA, 40% heptane
Gradient: isocratic
Flow Rate 1 ml/min

### Injection Volume: 5 μl

Column temperature 40° C.

### Detection 254 nm UV

Sample preparation: 5 mg in 10 ml of 60% IPA, 40% n-heptane
Retention time 7.5 min.

### Intermediate 3

### Ethyl (2R,4E)-7-chloro-4 (2-oxo-1-phenyl-3-pyrrolidinylidene)-1,2,3,4-tetrahydro-2-quinolinecarboxylate

Intermediate 3 (87 kg) triphenylphosphine (2.177 kg), palladium (II) chloride (0.589 kg) and silica gel (6.14 kg) were charged to toluene (1173 L). Triethylamine (22.3 kg) was added and the reagent washed in with toluene (45 L). The mixture was heated at reflux for 3 hours and 30 minutes when analysis of a sample (HPLC) indicated that the reaction was complete. Trimercaptotriazine (1.74 kg) was added and reflux continued for a further 1 hour. The solution was cooled to 45° C. and the solids (trimercaptotriazine/palladium complex and triethylamine hydrogeniodide) removed by filtration. The solids were washed with toluene (174 L).

With the temperature maintained at 40-45° C., the combined filtrates were washed twice with demineralised water (2×696 L) before being concentrated by distillation under reduced pressure to 696 L. The concentrate temperature was adjusted to 40° C. and iso-octane (870 L) was added over 77 minutes whilst maintaining the temperature at 41-43° C. The slurry was cooled and stirred at 15-17° C. for 2 hours. The title compound was isolated in a filter drier and washed with a 50:50 v/v mixture of toluene and isooctane (2×177.0 L) and dried in vacuo at 40-45° C. for 17 hours (48.5 kg).

¹H-NMR (CDCl₃) 8 (ppm) 7.68 (d, 2H, J=7.8 Hz), 7.39 (t, 2H, J=7.9 Hz), 7.16 (t, 1H, J=8.2 Hz), 7.13 (d, 1H, J=9.0 Hz), 6.66-6.63 (m, 2H), 4.77 (s, 1H), 4.20-4.04 (m, 4H), 3.85 (m, 1H), 3.77 (m, 1H), 3.44 (m, 1H), 3.15 (m, 2H), 1.17 (t, 3H, J=7.1 Hz).

### Chiral HPLC Column Chiralcel OD-H 250×4.6 mm.

Mobile Phase 0.1% TFA, 60% ethanol, 40% heptane
Gradient: isocratic for 20 minutes
Flow Rate 0.5 ml/min

### Injection Volume: 20 μl

Column temperature 30° C.

### Detection 254 nm UV

Sample preparation: 0.1 mg/ml in mobile phase
Retention time 12 min.

### EXAMPLE 1

### (2R,4E)-7-chloro-4-(2-oxo-1-phenyl-3-pyrrolidinylidene)-1,2,3,4-tetrahydro-2-quinolinecarboxylic acid, (2R,3R,4R,5S)-6-methylamino-hexane-1,2,3,4,5-pentanol (meglumine) salt (form 2)

Intermediate 3 (48.5 kg) was dissolved in a mixture of tetrahydrofuran (337 L) and demineralised water (160 L) and a 10% w/w sodium hydroxide solution (85.0 kg) [taken from a stock solution prepared from sodium hydroxide (39 kg) and demineralised water (390 L)], whilst maintaining the temperature at 1° C. The solution was washed in with demineralised water (10.0 L). The mixture was stirred at 0 to 1° C. for 4 hours. The reaction was deemed complete by HPLC analysis. 2.5M Hydrochloric acid (72 kg) was added, [taken from a stock solution prepared from concentrated hydrochloric acid (102.5 kg) and demineralised water (265 L)] maintaining the temperature at 1.5-3° C. The acid was washed in with demineralised water (10 L). Dichloromethane (388 L) was added and the phases separated. The aqueous phase was extracted with further dichloromethane (194 L), then the combined organic extracts were washed with demineralised water (194 L) before being filtered into a clean reactor and concentrated under vacuum to approximately 146 L. Filtered acetone (388 L) was added to the concentrate and the solution again concentrated under vacuum to 146 L.

The temperature of the solution concentrate was adjusted to 41° C. and a solution of N-methyl-D-glucamine (meglumine) (23.9 kg) in demineralised water (110 L) at 41° C. was added via a filter and the filter was washed with demineralised water (11.3 L). The resulting clear solution was diluted with filtered-acetone (97 L), the temperature adjusted to 42-C and seed crystals of title compound was added. Acetone (728 L) was added via a filter and the mixture stirred at 39.5° C. for 1 hour. Crystallisation occurred during the addition. The slurry was cooled to 22° C., stirred for 15 minutes, then cooled to 6° C. and stirred for a further 1 hour. The product was isolated in a filter drier, washed twice with acetone (2×97 L) and dried under vacuum at 35-40° C. for 36.5 hours to obtain the dried title compound (64.15 kg) which was passed through a 600 micron screen to break up any agglomerates to obtain the sieved title compound (62.6 kg)

¹H-NMR (D₂O) δ (ppm) 7.54-7.47 (m, 4H), 7.33-7.27 (m, 2H), 6.78 (s, 1H), 6.71 (d, 1H J=8.6 Hz), 4.09 (m, 1H), 3.95-3.73 (m, 7H), 3.67-3.62 (m, 2H), 3.23-3.07 (m, 5H), 2.76 (s, 3H).

Melting point 186° C.

### Chiral HPLC Column Chiralcel OJ, 10 um, 250×4.6 mm.

Mobile Phase 0.1% TFA, 80% ethanol, 20% heptane
Gradient: isocratic for 20 minutes
Flow Rate 1.0 ml/min

### Injection Volume: 20 μl

Column temperature 35° C.

### Detection 255 nm UV

Sample preparation: 0.1 mg/ml in mobile phase.
Retention time 9.4 min.

### Pharmacological Activity

The ability of the compound of formula (I) or pharmaceutically acceptable salts thereof to reduce nicotine craving in smokers was measured by means of self-administered questionnaires on Smoking Craving/Urge-Brief (QSU-Brief), as described by Tiffany S T, & Christen A G Cox L S, Nicotine and Tobacco Research, 3: 7-16, 2001.

The QSU-Brief is a specific craving scale that consists of 10 items scored on a scale from 1 to 7. The higher average score corresponds to the higher intensity of nicotine craving. The QSU-Brief has 2 factors: factor 1 refers to the anticipation of pleasure from smoke and factor 2 refers to anticipation of relief from negative affects of abstinence.

The study investigated nicotine craving in smokers abstinent for 3 days from cigarettes (enforced abstinence model) receiving either a single dose 120 mg iv of the compound of formula (I) as meglumine salt (Compound) or a single dose of placebo.

Self-administered questionnaires (QSU-Brief), were administered at pre-dose and 3, 6 and 12 hours post dose to evaluate craving.

As described in Table 1 smokers abstinent for 3 days and receiving the Compound had a significant average lower craving factor 2 than abstinent smokers receiving placebo.

TABLE 1
Average Craving Factor 2 during abstinence with
compound or placebo administration

| | Placebo | Compound |
|---|---|---|
| QSU-Brief: Craving Factor 2 | 4.0 | 3.6 |

A further study investigated nicotine craving in smokers abstinent for 72 hours from cigarettes (enforced abstinence model) receiving either repeated administrations of the compound of formula (I) as meglumine salt (Compound) or placebo. The Compound showed reduction in nicotine craving on 31 smoker volunteers in the double blind randomized, three-period, crossover study model. Each subject was exposed to 3 different experimental conditions:

1) Compound (120 mg/day) was administered intravenously for 7 days starting on Day 1 and quitted smoking abruptly on Day 5 for 72 hours (i.e. non-smoking period;)
2) Placebo was administered intravenously for 7 days starting on Day 1 and quitted smoking abruptly on Day 5 for 72 hours (i.e. non-smoking period;)
3) allowed to smoke for 7 consecutive days (i.e. free smoking period).

The self-administered questionnaire (QSU-Brief) was administered from day 5 to day 7, starting at 0 (8 a.m.) and then at 3, 6, 12, 24, 30, 36, 48, 54, 60 and 72 hours to evaluate craving.

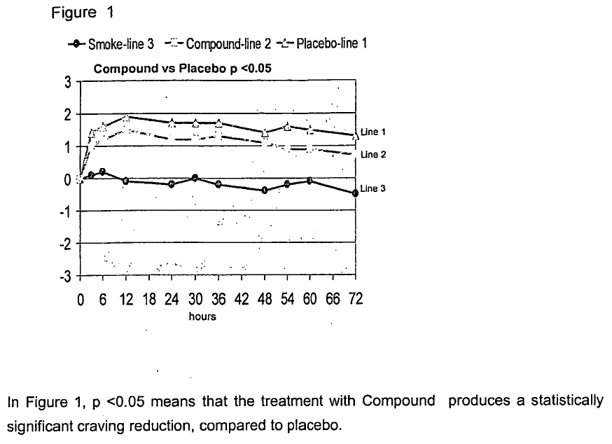

The results in FIG. 1 show that during the 72 hours of abstinence (X-axis) the Compound (line 2) reduced the nicotine craving level (QSU-Brief on Y axis) when compared to the Placebo (line 1). Craving level was significantly lower when the subjects were allowed to smoke (line 3) than during abstinence with placebo (line 1) or Compound (line2).

1. Use of a compound of formula (I) or pharmaceutically acceptable salts and solvates thereof 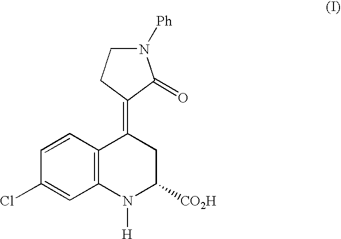
in the preparation of a medicament for use in the treatment of nicotine craving.

2. Use according to claim 1 wherein the pharmaceutically acceptable salt of the compound of formula (I) is meglumine.

3. A method for the treatment of nicotine craving comprising administering to that person an effective amount of a compound of formula (I) or pharmaceutically acceptable salts and solvates thereof.

4. Method for the treatment according to claim 3 wherein the pharmaceutically acceptable salt of the compound of formula (I) is meglumine.
